(19) United States
(12) Patent Application Publication
(10) Pub. No.: US 2018/0094854 A1
(43) Pub. Date: Apr. 5, 2018

(54) PROCESS AND PLANT FOR RECOVERING ARGON IN A UNIT FOR SEPARATING AN AMMONIA SYNTHESIS PURGE GAS

(71) Applicant: L'Air Liquide, Societe Anonyme pour I'Etude et I'Exploitation des Procedes Georges Claude, Paris (FR)

(72) Inventors: Bertrand Demolliens, Paris (FR); Antoine Hernandez, Pontault Combault (FR)

(73) Assignee: L'Air Liquide, Societe Anonyme pour I'Etude et I'Exploitation des Procedes Georges Claude, Paris (FR)

(21) Appl. No.: 15/720,015

(22) Filed: Sep. 29, 2017

(30) Foreign Application Priority Data
Oct. 3, 2016 (FR) FR 1659492

(51) Int. Cl. F25J 1/00; C01C 1/04; C01B 23/00; B01D 5/00; C01B 21/04

(52) U.S. Cl. CPC F25J 1/002; C01C 1/0476; C01B 23/0036; B01D 5/0075; C07C 7/11; F25J 1/0072; C01B 21/0433; C01B 2210/0034; B01D 5/0063

(57) ABSTRACT

A plant for producing an argon-rich stream from a mixture formed by a purge fluid from a plant for producing ammonia comprises at least two methane scrubbing columns upstream of a methane separation column and, downstream therefrom, a nitrogen/argon separation column.

### CROSS REFERENCE TO RELATED APPLICATIONS

This application claims the benefit of priority under 35 U.S.C. § 119 (a) and (b) to French patent application No. FR1659492, filed Oct. 3, 2016, the entire contents of which are incorporated herein by reference.

### FIELD OF THE INVENTION

The present invention relates to a process and to a plant for recovering argon in a unit for separating an ammonia synthesis purge gas.

### BACKGROUND

A conventional plant for producing ammonia by natural gas reforming generally comprises the following steps:
final desulfurization of the natural gas;
primary reforming;
postcombustion in air, with which the nitrogen for the synthesis is introduced;
conversion of CO;
decarbonatation;
methanization;
compression; and
ammonia synthesis loop.

In order to remove the inert species and prevent them accumulating in the system, the ammonia synthesis loop produces a purge gas that contains the following compounds: H₂, N₂, CH₄, Ar, NH₃. The mixture is substantially free of carbon monoxide but may or may not contain helium.

It may then be advantageous to treat this purge in a cryogenic unit in order to recover, on the one hand, the compounds that can be reused in the ammonia synthesis loop and, on the other hand, to produce argon in liquid form and to sell it.

“Production and Purification of Argon” by Arregger, Chemical and Process Engineering, October 1964, U.S. Pat. No. 4,338,108, “Cryogenic Gas Separation” by Duckett et al., The Chemical Engineer, December 1985, “Methods for argon recovery to meet increased demand on the argon market” by Springmann, AIChE Symposium Series 1982, U.S. Pat. No. 4,762,542, “Separation of Gases” by Isalski, pp. 84-88 and “Cryogenic Argon Recovery from Ammonia Plant Purge Gas” by Hwang et al., presented at “Cryogenics and Refrigeration”, Hangzhou, 1989 all disclose the use of a nitrogen scrubbing column in a process for the cryogenic separation of an ammonia synthesis purge gas in order to produce argon. This column is generally followed by an argon/methane separation column and a nitrogen/argon separation column.

It is also known to carry out a first step of partial condensation of the purge gas at low temperature in order to reduce investment of a nitrogen scrubbing column as described in U.S. Pat. No. 4,338,108. The argon extraction efficiency of such a plant is lower but considerably reduces the nitrogen cycle requirements.

It is also known to carry out several successive partial condensations (as described in FR 2 946 418) at several pressures. This makes it possible to increase the hydrogen recovery efficiency by integrating the cold-box process into the existing compressors of the ammonia unit.

FR 2 946 418 describes how the methane scrubbing in a scrubbing column makes it possible to lower the hydrogen content in the liquid phase at the bottom of the column 21. This also makes it possible to increase the argon recovery efficiency.

### SUMMARY OF THE INVENTION

According to one subject of the invention, provision is made to carry out successive scrubbings in order to increase the recovery efficiencies while only marginally increasing the unit investment.

According to one subject of the invention, a process is provided for producing an argon-rich stream from a mixture formed by a purge fluid from a plant for producing ammonia and that contains hydrogen, methane, nitrogen, argon and hydrogen in which:

i) the mixture is separated at low temperature in order to produce at least one hydrogen-enriched gas and one hydrogen-depleted liquid;

ii) at least one portion of the hydrogen-depleted liquid is sent to a main methane scrubbing column and a methane-enriched liquid is sent to the top of the methane scrubbing column in order to form an overhead gas and a bottom liquid;

iii) at least one portion of the bottom liquid from the main scrubbing column is sent to a methane separation column in order to produce a methane-enriched bottom liquid and a methane-depleted overhead gas;

iv) a portion of the methane-enriched bottom liquid constitutes the methane-enriched liquid sent to the top of the at least the main methane scrubbing column; and

v) at least one portion of the methane-depleted overhead gas is sent to a nitrogen/argon separation column in order to form a nitrogen-enriched fluid at the top of the column and an argon-rich liquid at the bottom of the column that is used as product,

wherein the separation of step i) is carried out by at least one methane scrubbing step in at least one auxiliary methane scrubbing column supplied at the top by another portion of the methane-enriched bottom liquid.

According to other optional features:
the separation of step i) is carried out by partial condensation and by scrubbing in which the liquid from the partial condensation is sent to an auxiliary methane scrubbing column, the bottom liquid of which constitutes the hydrogen-depleted liquid;
the separation of step i) is carried out by partial condensation and by scrubbing in which the liquid from an auxiliary methane scrubbing column is sent to the partial condensation, the bottom liquid of which constitutes the hydrogen-depleted liquid;
the separation of step i) is carried out by scrubbing in a first auxiliary methane scrubbing column and a second auxiliary methane scrubbing column, the bottom liquid from the first auxiliary methane scrubbing column being sent to the second auxiliary methane scrubbing column and the bottom liquid from the second auxiliary methane scrubbing column constituting the hydrogen-depleted liquid;
at least one portion of the methane-enriched bottom liquid is pressurized, divided into two and sent to the top of the main scrubbing column and to the top of at least one auxiliary scrubbing column;
the auxiliary scrubbing column(s) operate(s) at a lower pressure than the main scrubbing column;
the phase separator produces a hydrogen-enriched gas at higher or lower pressure than the hydrogen-enriched gas produced by the auxiliary methane scrubbing column;
the first auxiliary scrubbing column produces a hydrogen-enriched gas at higher pressure than the second auxiliary scrubbing column;
the methane-enriched liquid is pressurized upstream of the methane scrubbing column; and/or
the process is kept cold at least partially by a nitrogen cycle.

According to another aspect of the invention, a plant is provided for producing an argon-rich stream from a mixture formed by a purge fluid from a plant for producing ammonia and that contains hydrogen, methane, nitrogen, argon and hydrogen, comprising:

i) at least one low-temperature separation column, supplied by the purge fluid or a fluid derived from the purge fluid, for producing at least one hydrogen-enriched gas and one hydrogen-depleted liquid;

ii) a main methane scrubbing column, a line for sending at least one portion of the hydrogen-depleted liquid to the main methane scrubbing column, and a line for sending a methane-enriched liquid to the top of the methane scrubbing column in order to form an overhead gas and a bottom liquid;

iii) a methane separation column, a line for sending at least one portion of the bottom liquid from the main scrubbing column to the methane separation column in order to produce a methane-enriched bottom liquid and a methane-depleted overhead gas;

iv) a portion of the methane-enriched bottom liquid constituting the methane-enriched liquid sent to the top of the main methane scrubbing column; and

v) a nitrogen/argon separation column, at least one portion of the methane-depleted overhead gas is sent to the nitrogen/argon separation column in order to form a nitrogen-enriched fluid at the top of the column and an argon-rich liquid at the bottom of the column that is used as product,

wherein the at least one low-temperature separation column of step i) is at least one methane scrubbing column connected to the bottom of the methane separation column.

The plant optionally comprises a single pump connected to the bottom of the methane separation column and at least one main methane scrubbing column and at least one auxiliary methane scrubbing column.

### BRIEF DESCRIPTION OF THE DRAWINGS

The present invention will be clearly understood and its advantages will also become evident in the light of the description which will follow, given solely by way of non-limiting example and made with reference to the attached drawings, in which:

FIG. 1 provides an embodiment of the present invention.

FIG. 2 provides an additional embodiment of the present invention.

FIG. 3 provides another embodiment of the present invention.

### DETAILED DESCRIPTION OF THE INVENTION

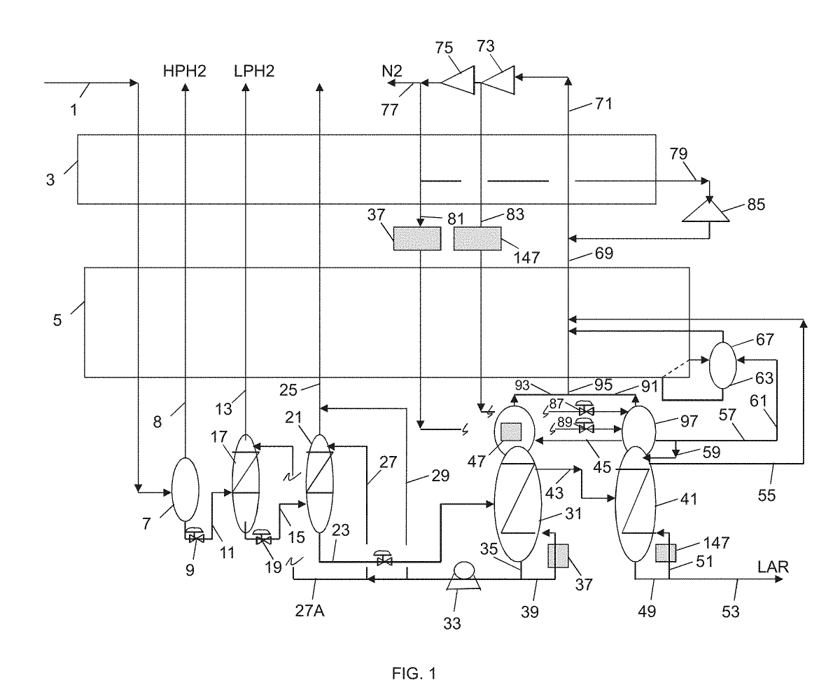

What is considered in certain embodiments of the invention is to increase the number of stages of the methane-pressurizing pump 33 in order to be able to carry out methane scrubbing in place of the separator vessel 7 and/or the separator vessel 17 from FIG. 1 of FR-A-2946418.

The methane scrubbing in a column in place of the separator vessel 7 makes it possible to considerably improve the argon yield of the process. Specifically, the argon that left the process by thermodynamic equilibrium in the fluid 8 is now recovered and separated, which increases the argon extraction efficiency by 20 to 25%.

The methane scrubbing in a column in place of the separator vessel 17 is slightly less advantageous since the stream 13 is already quite low but increases the argon extraction efficiency by around +2 to 3%.

It could be advantageous to raise the pressure of the methane 35 by means of a single pump directly to the pressure of the column 7 then to expand this fluid for the methane scrubbing(s) at lower pressure, or else by two pumps in series, one operating from the pressure of the column up to the pressure of the fuel gas networks and the other from the pressure of the fuel gas network to the scrubbing pressure.

It is interesting to see that if the pump 33 no longer operates, the plant still operates but with a reduced argon yield (similar to what it would have with a partial condensation scheme). It is therefore possible to choose not to double up this pump. If the pressure of the fuel gas network is too high to send the methane into this network, the methane will be flared and a makeup of another fuel will have to be used in the fuel gas network to compensate for the missing heat energy.

The invention will be described in greater detail by referring to the figures which illustrate a plant according to the invention.

According to FIG. 1, a mixture of hydrogen, methane, nitrogen and argon 1, substantially free of carbon monoxide and preferably substantially free of helium, is cooled in a first heat exchanger 3 and then in a second heat exchanger 5 where it partially condenses. The partially condensed stream is sent to a first phase separator 7. The hydrogen-enriched gas 8 from the first phase separator is sent to the two exchangers 5, 3 to be reheated therein. The liquid from the first phase separator is expanded in a valve 9 in order to partially vaporize and the partially vaporized stream 11 is sent to an auxiliary methane scrubbing column 17. The auxiliary methane scrubbing column 17 is supplied at the top by a stream 27A of liquid methane originating from the pump 33 at the same pressure as the stream 27. The hydrogen-enriched gas 13 from the methane scrubbing column is sent to the two exchangers 5, 3 to be reheated therein. The liquid from the auxiliary scrubbing column 17 is expanded in a valve 19 in order to partially vaporize and the partially vaporized stream 15 is sent to the bottom of a methane scrubbing column 21 supplied at the top by a stream 27 of liquid methane also originating from the pump 33.

The pump 33 is not connected to another substantially identical pump connected in parallel with it.

The overhead gas from the methane scrubbing column is mixed with a stream 29 of pressurized liquid methane, originating from the pump 33, in order to form the stream 25 and the stream 25 is reheated in the two exchangers 5, 3 in order to form a fuel gas. The bottom liquid 23 is expanded in a valve then sent to an intermediate level of a methane separation column 31. The bottom liquid 35 of this column 31 is pressurized by a pump 33 and sent partly (stream 27) to the top of the scrubbing column 21 and partly (stream 29) mixed with the overhead gas of the scrubbing column. The column 31 has a bottom reboiler 37 supplied by a bottom stream 39 from the column 31. The column 31 also has an overhead condenser 47 where the overhead gas enriched in nitrogen and in argon is condensed. An overhead gas stream 43 is sent to an intermediate level of the column 41. The column 41 has a bottom reboiler 147 and an overhead nitrogen storage tank 97. The bottom liquid 49 is sent partly (stream 51) to the bottom reboiler 147 and the rest 53 serves as argon-rich liquid product. The nitrogen-rich overhead gas 55 from the column 41 is sent to an intermediate level of the exchanger 5.

A nitrogen cycle ensures the reboiling of the columns 31, 41 and the cooling of the overhead condenser 47 and provides the cooling of the top of the column 41 by direct reflux. Nitrogen 71 is compressed in a compressor 73 and divided into two. One portion is sent to the compressor 75 in order to form the high-pressure nitrogen product 77 and a high-pressure cycle stream. The cycle stream is cooled to an intermediate temperature of the exchanger 3 then is divided into two. One portion 1 is used to heat the reboiler 37 then is sent to the storage tank through the valve 89. Another portion 79, at an intermediate temperature of the exchanger 3, is expanded in a turbine 85 and mixed with the stream 69 in order to form the stream 71. The nitrogen from the compressor 73 is used for reboiling the reboiler 147 as stream 83, then is expanded by the valve 87 and sent to the storage tank 97. The liquid from the storage tank is withdrawn as two streams, a stream 47 being sent to the overhead condenser 47 and the other stream 57 being partly (59) sent back to the column 41 and partly (61) sent to a phase separator 63. The gas from the phase separator is mixed with streams 93, 91 coming respectively from the overhead condenser 47 and from the storage tank 97. This mixed stream is reheated in the exchanger 5, is mixed with the stream 55 and forms the stream 69. The liquid 65 from the phase separator 63 is reheated in the exchanger 5.

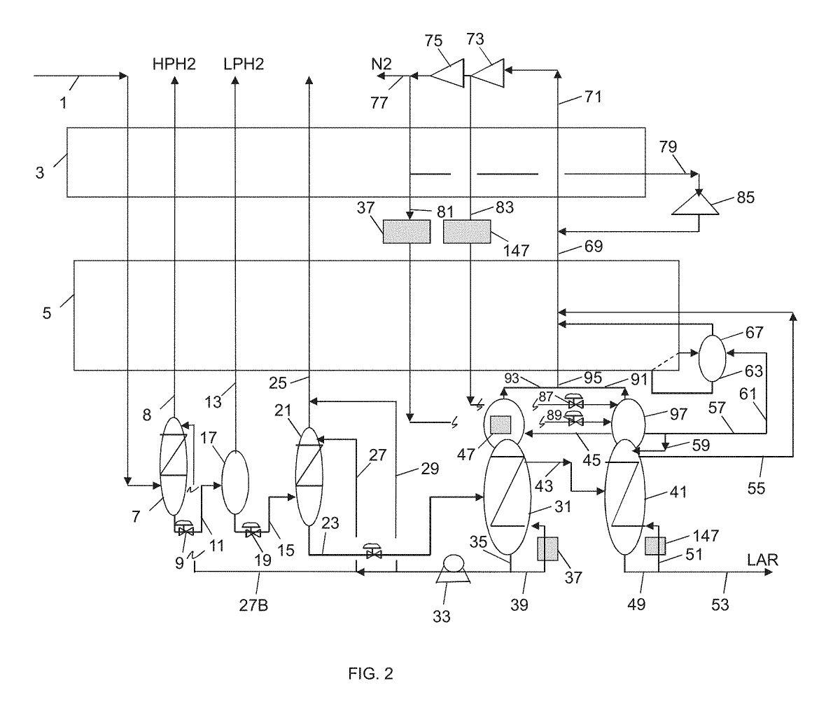

In FIG. 2, the order of the phase separator and of the auxiliary methane scrubbing column of FIG. 1 is reversed. In FIG. 2, a mixture of hydrogen, methane, nitrogen and argon 1, substantially free of carbon monoxide and preferably substantially free of helium, is cooled in a first heat exchanger 3 and then in a second heat exchanger 5 where it partially condenses. The partially condensed stream is sent to an auxiliary methane scrubbing column 7 supplied at the top by a stream 27B of liquid methane originating from the pump 33 at the same pressure as the stream 27. The hydrogen-enriched gas 8 from the auxiliary methane scrubbing column 7 is sent to the two exchangers 5, 3 to be reheated therein. The liquid from the auxiliary methane scrubbing column 7 is expanded in a valve 9 in order to partially vaporize and the partially vaporized stream 11 is sent to a first phase separator 17. The hydrogen-enriched gas 13 from the first phase separator is sent to the two exchangers 5, 3 to be reheated therein. The liquid from the first phase separator 17 is expanded in a valve 19 in order to partially vaporize and the partially vaporized stream 15 is sent to the bottom of a methane scrubbing column 21 supplied at the top by a stream 27 of liquid methane also originating from the pump 33.

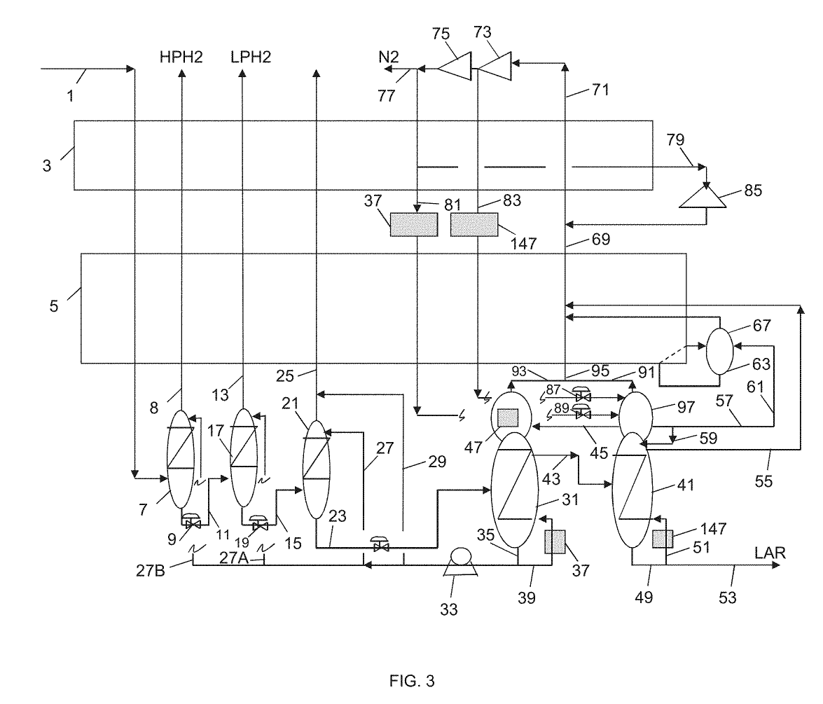

FIG. 3 differs from FIG. 1 in that there are not two but three methane scrubbing columns in series (at least one auxiliary methane scrubbing column and one main methane scrubbing column) and no phase separator upstream of the methane scrubbing columns. In FIG. 3, a mixture of hydrogen, methane, nitrogen and argon 1, substantially free of carbon monoxide and preferably substantially free of helium, is cooled in a first heat exchanger 3 and then in a second heat exchanger 5 where it partially condenses. The partially condensed stream is sent to a first auxiliary methane scrubbing column 7 supplied at the top by a stream 27B of liquid methane originating from the pump 33 at the same pressure as the stream 27. The hydrogen-enriched gas 8 from the first auxiliary methane scrubbing column 7 is sent to the two exchangers 5, 3 to be reheated therein. The liquid from the first auxiliary methane scrubbing column 7 is expanded in a valve 9 in order to partially vaporize and the partially vaporized stream 11 is sent to a second methane scrubbing column 17. The hydrogen-enriched gas 13 from the second auxiliary methane scrubbing column 17 is sent to the two exchangers 5, 3 to be reheated therein. The liquid from the second auxiliary methane scrubbing column 17 is expanded in a valve 19 in order to partially vaporize and the partially vaporized stream 15 is sent to the bottom of a third methane scrubbing column 21 supplied at the top by a stream 27 of liquid methane also originating from the pump 33.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

What is claimed is:

1. A process for producing an argon-rich stream from a mixture formed by a purge fluid from a plant for producing ammonia and that contains methane, nitrogen, argon and hydrogen, wherein the method comprises the steps of:
i) separating the mixture at a sub-ambient temperature in order to produce at least one hydrogen-enriched gas and one hydrogen-depleted liquid;
ii) sending at least one portion of the hydrogen-depleted liquid to a main methane scrubbing column while introducing a methane-enriched liquid to the top of the methane scrubbing column in order to form an overhead gas and a bottom liquid;
iii) sending at least one portion of the bottom liquid from the main scrubbing column to a methane separation column in order to produce a methane-enriched bottom liquid and a methane-depleted overhead gas, wherein a portion of the methane-enriched bottom liquid constitutes the methane-enriched liquid sent to the top of the at least the main methane scrubbing column in step ii); and
iv) sending at least one portion of the methane-depleted overhead gas to a nitrogen/argon separation column in order to form a nitrogen-enriched fluid at the top of the column and an argon-rich liquid at the bottom of the column that is used as product,
wherein the separation of step i) is carried out by at least one methane scrubbing step in at least one auxiliary methane scrubbing column supplied at the top by another portion of the methane-enriched bottom liquid.

2. The process according to claim 1, in which the separation of step i) is carried out by partial condensation and by scrubbing in which the liquid from the partial condensation is sent to an auxiliary methane scrubbing column, the bottom liquid of which constitutes the hydrogen-depleted liquid.

3. The process according to claim 2, in which the phase separator produces a hydrogen-enriched gas at higher or lower pressure than the hydrogen-enriched gas produced by the auxiliary methane scrubbing column.

4. The process according to claim 1, in which the separation of step i) is carried out by partial condensation and by scrubbing in which the liquid from an auxiliary methane scrubbing column is sent to the partial condensation, the bottom liquid of which constitutes the hydrogen-depleted liquid.

5. The process according to claim 1, in which the separation of step i) is carried out by scrubbing in a first auxiliary methane scrubbing column and a second auxiliary methane scrubbing column, the bottom liquid from the first auxiliary methane scrubbing column being sent to the second auxiliary methane scrubbing column and the bottom liquid from the second auxiliary methane scrubbing column constituting the hydrogen-depleted liquid.

6. The process according to claim 5, in which the first auxiliary scrubbing column produces a hydrogen-enriched gas at higher pressure than the second auxiliary scrubbing column.

7. The process according to claim 1, in which at least one portion of the methane-enriched bottom liquid is pressurized, divided into two and sent to the top of the main scrubbing column and to the top of at least one auxiliary scrubbing column.

8. The process according to claim 1, in which the auxiliary scrubbing column(s) operate(s) at a lower pressure than the main scrubbing column.

9. The process according to claim 1, in which the methane-enriched liquid is pressurized upstream of the methane scrubbing column.

10. The process according to claim 1, further comprising a nitrogen cycle configured to provide a portion of refrigeration used by the process.

11. A plant for producing an argon-rich stream from a mixture formed by a purge fluid from a plant for producing ammonia and that contains methane, nitrogen, argon and hydrogen, the plant comprising:
i) at least one low-temperature separation column, supplied by the purge fluid or a fluid derived from the purge fluid, for producing at least one hydrogen-enriched gas and one hydrogen-depleted liquid;
ii) a main methane scrubbing column, a line for sending at least one portion of the hydrogen-depleted liquid to the main methane scrubbing column, and a line for sending a methane-enriched liquid to the top of the methane scrubbing column in order to form an overhead gas and a bottom liquid;
iii) a methane separation column, a line for sending at least one portion of the bottom liquid from the main scrubbing column to the methane separation column in order to produce a methane-enriched bottom liquid and a methane-depleted overhead gas;
iv) a portion of the methane-enriched bottom liquid constituting the methane-enriched liquid sent to the top of the main methane scrubbing column; and
v) a nitrogen/argon separation column, at least one portion of the methane-depleted overhead gas is sent to the nitrogen/argon separation column in order to form a nitrogen-enriched fluid at the top of the column and an argon-rich liquid at the bottom of the column that is used as product,
wherein the at least one low-temperature separation column of step i) is at least one methane scrubbing column connected to the bottom of the methane separation column.

12. The plant according to claim 11, comprising a single pump connected to the bottom of the methane separation column and at least one main methane scrubbing column and at least one auxiliary methane scrubbing column.
